(19) United States
(12) Patent Application Publication
(10) Pub. No.: US 2023/0078148 A1
(43) Pub. Date: Mar. 16, 2023

(54) INDEXING SPATIAL INFORMATION FOR A SINGLE-CELL DOWNSTREAM APPLICATIONS

(71) Applicant: Yeda Research and Development Co. Ltd., Rehovot (IL)

(72) Inventors: Amos TANAY, Rehovot (IL); Elad CHOMSKY, Rehovot (IL); Yonatan STELZER, Rehovot (IL); Raz BEN-YAIR, Rehovot (IL)

(73) Assignee: Yeda Research and Development Co. Ltd., Rehovot (IL)

(21) Appl. No.: 17/990,770

(22) Filed: Nov. 21, 2022

Related U.S. Application Data
(63) Continuation of application No. PCT/IL2021/050575, filed on May 19, 2021.
(60) Provisional application No. 63/086,100, filed on Oct. 1, 2020.

(30) Foreign Application Priority Data
May 20, 2020 (IL) 274811

(51) Int. Cl. G01N 33/58; G01N 1/30; G06V 20/69

(52) U.S. Cl. CPC G01N 33/582; G01N 1/30; G06V 20/69

(57) ABSTRACT

A method of identifying a molecular composition and a spatial position of a single cell comprised in a 3 dimensional structure comprising a plurality of cells is provided.

### RELATED APPLICATIONS

This application is a Continuation (CON) of PCT Patent Application No. PCT/IL2021/050575 having International filing date of May 19, 2021, which claims the benefit of priority under 35 USC § 119(e) of U.S. Provisional Patent Application No. 63/086,100 filed on Oct. 1, 2020 and of Israel Patent Application No. 274811 filed on May 20, 2020. The contents of the above applications are all incorporated by reference as if fully set forth herein in their entirety.

### FIELD AND BACKGROUND OF THE INVENTION

The present invention, in some embodiments thereof, relates to indexing spatial information for a single-cell downstream applications.

In recent years, single-cell technologies have revolutionized our understanding of cellular heterogeneity and are extensively utilized in both basic science and biomedical research. However, a key obstacle that persists, is determining the original location and context of single cells prior to their unavoidable dissociation into cell suspensions. This drawback impedes our ability to study cell-cell interactions and tissue-level architecture, thus limiting our understanding of cell function in the context of its immediate environment. While significant efforts have been made to overcome this key challenge¹⁻⁸, current methods are limited in several aspects:

(i) Methods that utilized RNA FISH typically require a-priory knowledge of landmark genes that can serve as location identifiers⁹⁻¹¹. Furthermore, such methods are limited to tissues harboring previously resolved organizations, such as intrinsic gradients. Recent development in multiplexed spatial single-cell transcriptomics are still far from providing the needed flexibility and simplicity to be routinely used and for analysis of a large number of samples and various perturbations^{5,12-14}.

(ii) While it is possible to surgically dissect or slice tissues into defined fragments, this approach is highly labor-intensive, making progressive analysis through complex tissues and various samples essentially unfeasible¹⁵.

(iii) Although it is possible to use endogenous reporters to resolve spatial organization, this approach requires using transgenic strains that provide spatial information limited to cells expressing the reporter¹⁶.

(iv) Finally, all current methods used to-date are transcription-based and do not allow spatial epigenetics, proteomics or metabolomics.

### SUMMARY OF THE INVENTION

According to an aspect of some embodiments of the present invention there is provided a method of identifying a molecular composition and a spatial position of a single cell comprised in a 3 dimensional (3D) structure comprising a plurality of cells, the method comprising:

(a) injecting into the 3D structure at least one dye to form an identifiable color pattern of the at least one dye over the plurality of cells;
(b) identifying the color pattern in the 3D structure so as to index the plurality of cells according to spatial positions of the cells in the 3D structure;
(c) isolating cells from the 3D structure comprising the at least one dye to obtain single cells;
(d) determining a molecular composition and staining of the single cells;
(e) aligning the staining of the single cells to the index so as to identify the spatial position of the single cells.

According to some embodiments of the invention, the color pattern comprises a diffusion gradient.

According to some embodiments of the invention, the color pattern comprises a plurality of colors, each characterized by a different hue or central wavelength.

According to some embodiments of the invention, the plurality of cells comprise a tissue.

According to some embodiments of the invention, the plurality of cells comprise an organoid.

According to some embodiments of the invention, the plurality of cells comprise an organ.

According to some embodiments of the invention, the plurality of cells comprise an organism.

According to some embodiments of the invention, the 3D structure comprises a gel embedding the plurality of cells.

According to some embodiments of the invention, the infecting is into the gel.

According to some embodiments of the invention, the injecting is into the plurality of cells.

According to some embodiments of the invention, the dye is at least one of:

(i) non-toxic;
(ii) membrane permeable;
(iii) capable of forming staining diffusion gradient; and
(iv) not leaking from cells following the isolating.

According to some embodiments of the invention, the dye is a nucleic acid binding dye.

According to some embodiments of the invention, the dye is selected from the group consisting of Syto13, Syto41 and Syto60.

According to some embodiments of the invention, the staining diffusion gradient is obtained by a plurality of dyes.

According to some embodiments of the invention, the staining diffusion gradient is obtained by varying concentrations of the at least one dye.

According to some embodiments of the invention, staining diffusion gradient is an opposing gradient or coalescing gradient.

According to some embodiments of the invention, the staining diffusion gradient is a radial gradient.

According to some embodiments of the invention, the method further comprises data mining a suggested structure of the plurality of cells prior to step (a).

According to some embodiments of the invention, the isolating is by enzymatic dissociation.

According to some embodiments of the invention, the determining the molecular composition of the single cells is selected from a transcriptome, a proteome, a peptidome, a metabolome.

According to some embodiments of the invention, the molecular composition is determined by a method selected from the group consisting of an RNAseq, ChIPseq, BSseq, and ATACseq.

According to an aspect of some embodiments of the present invention there is provided a method of identifying a position of a single cell in an image of cells, the method comprising: receiving a staining characteristic of the single cell;

identifying a color pattern in the image, so as to index the cells according to spatial positions of the cells in image; and

aligning the staining of the single cell to the index so as to identify the spatial position of the single cell.

According to some embodiments of the invention, the color pattern comprises a diffusion gradient.

According to some embodiments of the invention, the color pattern comprises a plurality of colors, each characterized by a different hue or central wavelength.

According to some embodiments of the invention, the identifying the color pattern, comprises binning the image into a plurality of spatial bins, and estimating a relative abundance of cells in each spatial bin.

According to some embodiments of the invention, the estimating the relative abundance is based on confocal microscopy data.

According to some embodiments of the invention, the cells form a tissue having a structure, and wherein the estimating the relative abundance is based on the structure.

According to some embodiments of the invention, the identifying the color pattern, comprises thresholding picture-elements in the image, to binary classify each picture-element as stained or non-stained.

According to some embodiments of the invention, the thresholding is based on an estimated number of cells per staining characteristic in the image.

According to some embodiments of the invention, the method is executed for a plurality of cell types, wherein the aligning comprises estimating a likelihood for the plurality of cell types to have a respective plurality of staining characteristics.

According to some embodiments of the invention, the method further comprises applying an optimization procedure to the estimated likelihood.

According to some embodiments of the invention, the optimization procedure is a non-linear optimization procedure.

According to some embodiments of the invention, the optimization procedure comprises at least one of: a steepest descent procedure, conjugate-gradients procedure, and a quasi-Newton procedure.

According to some embodiments of the invention, the optimization procedure comprises a Broyden-Fletcher-Goldfarb-Shanno (BFGS) procedure, preferably an L-BFGS procedure.

According to some embodiments of the invention, the optimization procedure comprises Monte-Carlo simulation.

According to an aspect of some embodiments of the present invention there is provided a computer software product, comprising a computer-readable medium in which program instructions are stored, which instructions, when read by a data processor, cause the data processor to receive an image of cells, and a staining characteristic of a single cell, and to execute the method as described herein.

According to an aspect of some embodiments of the present invention there is provided a system for identifying a position of a single cell in an image of cells, the system comprising:

an input circuit receiving an image of cells, and a staining characteristic of the single cell; and

a data processor configured for executing the method as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an overview of experimental design. Spatial information is reconstructed in conjunction with whole-genome data derived from single mouse embryo cells using directional dye staining that generates a graded intensity pattern. Spatial position is inferred from signal intensity using a combination of FACS analysis (right) and fluorescent imaging (left).

FIGS. 2A, 2B, 2C, 2D and 2E show the characterization of Syto dye diffusion. (A) Dye screen in zebrafish embryos: tail region of 48 hpf embryos embedded in matrigel droplets. Syto dyes were injected into the matrigel adjacent to the tails and allowed to diffuse into the tissue. Appropriate dyes were subsequently selected based on diffusion properties. (B-C) overlaid FACS plots depicting fluorescent signal peaks from mES cohorts stained with a series of concentrations of Syto60 (red) and Syto12 (green), as well as unstained samples. (D-E) Plots derived from the corresponding pooled samples, 1 hour after pooling. Syto12 is leaky, demonstrated by merging of the signal peaks, while Syto60 is retained. Unstained samples were not included.

FIGS. 3A-3B show a toxicity assay for selected Syto dyes. (A) Heatmap of Pearson correlation between RNAseq samples derived from mESC cohorts stained with Syto 13, 41 and 60, 1 hr post staining, according to gene expression values. (B) Clustering dendrogram of the same samples according to gene expression using Ward's minimum variance method.

FIG. 4 shows an alternative application of dyes. Depicted are two potential methods of injections to (left) generate fluorescent gradient signal in intact tissues and (right) for color-coding discrete cell populations, both in intact tissues or following dissociation into single cells.

FIGS. 5A, 5B and 5C show the characterization of dye diffusion in mouse embryos. (A) Examples of E7.5 mouse embryos stained in a directional manner using a single dye (left) or a combination of two dyes (right). (B) Example of two dye staining in embryos at E8.5. Radial gradients are marked in white. FACS analysis showing fluorescent signal corresponding to the spatial radial gradient of the dyes. (C) Shown are mESCs color-coded using 2 dyes with 3 concentrations each, thus generating 9 different combinations. Note that dyes are spectrally compatible with the fluorescent proteins GFP and tdTomato.

FIGS. 6A, 6B, 6C, 6D, 6E and 6F show dye based spatial mapping of the E7.5 embryo. (A) The known structure of the E7.5 embryo (taken from [¹⁹]). A: Anterior. AVE: Anterior visceral endoderm. DEnd: Definitive endoderm. Dist: Distal. EXE: Extraembryonic ectoderm. EXM/En: Extraembryonic mesoderm/endoderm. ME: Mesoderm. N: Node. NE: Neurectoderm. P: Posterior. Prox: Proximal. SE: Surface ectoderm. (B) Dyed E7.5 embryo. Fluorescent dyes were injected directly into the embryo, resulting in small, well localized fluorescent loci. (C) Dyed E7.5 embryo. Fluorescent dyes were injected into the Matrigel adjacent to the embryo, resulting in a diffuse fluorescent signal. (D) Partition of the E7.5 embryo, without extraembryonic components, into 9 spatial bins (sbins). (E) Annotation of the spatial bins over an embryo's image. The rostral (anterior) and caudal (posterior) ends of the embryo are marked by the letters R and C respectively. The same embryo is presented as in (B). (F) The red fluorescent channel in an embryo's image (top) and the pixels that were identified as fluorescing in red (bottom). The same embryo is presented as in (B).

FIGS. 7A, 7B, 7C, 7D, 7E, 7F, 7G, 7H, 7I, 7J, 7K, and 7L show results of dye based spatial mapping. (A-L) Spatial mapping of selected cell types of the E7.5 mouse embryo. Scale bars show fold-change compared to the fraction of all cells contained within the sbin, as measured by confocal microscopy.

FIG. 8 is a schematic illustration of a system suitable identifying a position of a single cell in an image of cells, according to some embodiments of the present invention.

### DESCRIPTION OF SPECIFIC EMBODIMENTS OF THE INVENTION

The present invention, in some embodiments thereof, relates to indexing spatial information using direct dye labeling for a single-cell downstream applications.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst conceiving embodiments of the invention, the present inventors address the challenge of aligning whole-genome datasets derived from single cells with their spatial position in an unbiased and flexible manner. To this end, a novel method has been developed that uses fluorescent dyes to “index” spatial information in tissues. The experimental paradigm incorporates existing materials with novel computational methodologies in order to directly label, document and associate cell positions in a manner compatible with downstream single-cell molecular applications such as RNAseq, ChIPseq, BSseq, and ATACseq. Furthermore, the approach of using fluorescent dyes to gain spatial information is unbiased and does not rely on a-priory landmark genes, e.g., markers or endogenous reporters. These unique features make this approach adaptable to many systems, large numbers of samples, and mutants. It is envisaged that this technology can be easily and immediately applicable for enhancing research in any animal model, including mouse and other. In particular, when a tissue under study can be sampled repeatedly, embodiments of the present methods can have essentially unlimited resolution. Finally, it is anticipated that translation of the present approach to resolving three-dimensional structures in the context of single cell pathology analysis, where staining of multiple pathological slides can provide rich spatial registration of diverse cell types, including tumor cells, immune and stromal cells.

As is detailed below and in the Examples section which follows, the development of a dye method and associated algorithms that allow inferring positions of cell types in complex tissues are described herein. This method is applied to reconstruct 3D spatial organization of the post-implantation mouse embryo.

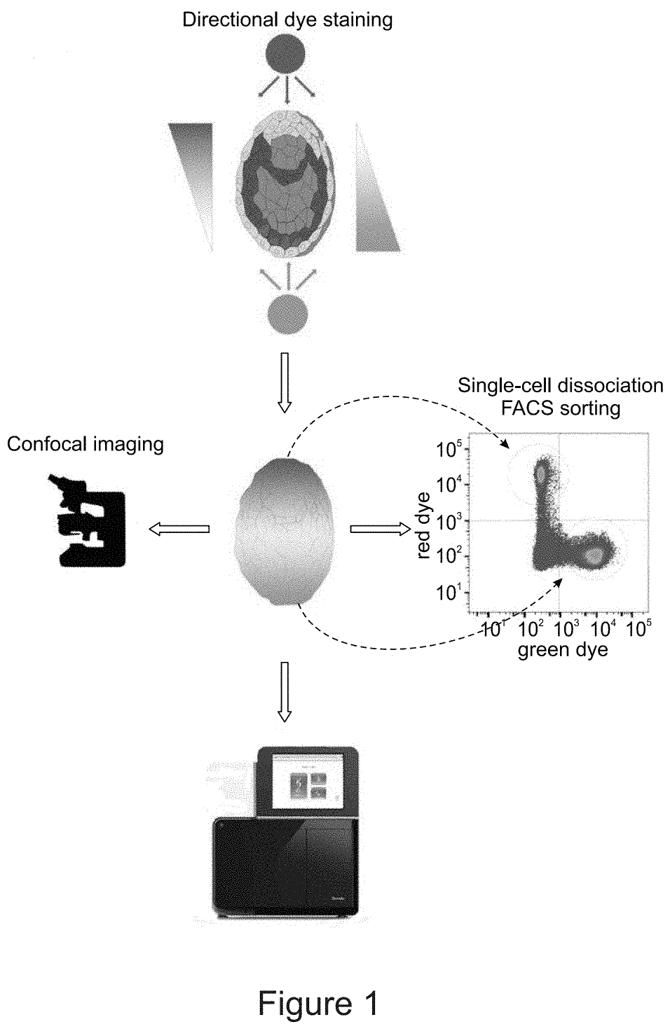

Thus, to associate single cell RNA-seq (scRNA-seq) profiles with their original localization within a tissue, fluorescent dyes are applied to intact specimens and form diffusion gradients around one or more focal points under the microscope. It is suggested that if dyes stain cells in an inert but still stable fashion, the graded and diffused signal that will be formed in a tissue of interest could be recovered during single cell FACS sorting. In this way, sequenced scRNA-seq profiles can be associated with partial information regarding their spatial source, allowing computational models to combine data from multiple cells and diffusion gradient and reconstruct a coherent transcriptional landscape of single cells in the tissue (FIG. 1).

Thus, according to an aspect of the invention there is provided a method of identifying a molecular composition and a spatial position of a single cell comprised in a 3 dimensional (3D) structure comprising a plurality of cells, the method comprising:

(a) injecting into the 3D structure at least one dye to form an identifiable color pattern of the at least one dye over the plurality of cells;
(b) identifying the color pattern in the 3D structure so as to index the plurality of cells according to spatial positions of the cells in the 3D structure;
(c) isolating cells from the 3D structure comprising the at least one dye to obtain single cells;
(d) determining a molecular composition and staining of the single cells;
(e) aligning the staining of the single cells to the index so as to identify the spatial position of the single cells.

It will be appreciated that the method may further comprise data mining (from the literature or even dedicated experiments) a suggested structure for the plurality of cells in the 3D structure prior. This can be done prior to the infection(s) and may facilitate in determining the position of injection, and various other parameters such as number of dyes, types, concentrations and the like.

As used herein “molecular composition” refers to a composition that can be analyzed by single cell analysis including but not limited to single cell genomics (DNA), transcriptomics (RNA), proteomics (peptides and proteins) or metabolomics (chemicals e.g., small molecules).

Such single cell analyses can be done using methods which are well known in the art, some of which are listed below by way of example and in the Examples section which follows.

As used herein “spatial position” refers to the position of a single in space, specifically in a specimen of a 3D structure.

As used herein “3 dimensional structure comprising a plurality of cells” refers to a cellular structure comprising a plurality of cells (e.g., more than 10 cells) forming a cell aggregate, a tissue culture, a tissue, an organoid, an organ, or even a whole organism.

As used herein “cell aggregate” refers to cells which are grouped together, not tightly joined and thus not forming tissues and do not comprise the distinct structures that are present in tissues e.g., vasculature. According to a specific embodiment, the cell aggregate comprise a single type of cells or more e.g., 2, 3, 4, 5.

Cell aggregates are important tools in the study of tissue development, permitting correlation of cell-cell interactions with cell differentiation, viability and migration, as well as subsequent tissue formation. The aggregate morphology permits re-establishment of the cell-cell contacts normally present in tissues; therefore, cell function and survival are often enhanced in aggregate culture. Because of this, cell aggregates may also be useful in tissue engineering, enhancing the function of cell-based hybrid artificial organs or reconstituted tissue transplants.

As used herein “tissue culture” refers to a cell culture which can be a monolayer but in any case refers to an adherent culture. Though typically referred to a two dimensional structures, monolayers are actually of a 3D structure.

As used herein “tissue” refers to a cellular organizational level between cells and a complete organ. A tissue is an ensemble of similar cells and their extracellular matrix from the same origin that together carry out a specific function. Organs are then formed by the functional grouping together of multiple tissues. The tissue typically comprises vasculature.

As used herein “organoid” refers to a miniaturized and simplified version of an organ produced in vitro in three dimensions that shows realistic micro-anatomy. They are derived from one or a few cells from a tissue, embryonic stem cells or induced pluripotent stem cells, which can self-organize in three-dimensional culture owing to their self-renewal and differentiation capacities. Organoids are used by scientists to study disease and treatments in a laboratory.

As used herein “organ” refers to a group of tissues that have been adapted to perform a specific function in a living organism. For example, developing embryonic tissues, the developing brain, bone marrow, colon, various tumor tissues and other spatially structures within tissues.

As used herein “organism” refers to any animal or human at any developmental stage (e.g., embryo, fetus, adult).

When the 3D structure is reproducible (e.g., healthy organ), the method may analyze a plurality of such structures.

When the 3D structure is unique (e.g., tumor), the 3D structure is analyzed by multiple sections of a single 3D structure.

According to a specific embodiment, the organism is an animal model.

According to a specific embodiment, the organism is a zebrafish.

According to a specific embodiment, the organism is a mouse.

Also included under these definitions portions of these structures e.g., tissue sections.

According to other embodiments, the 3D structure is unsectioned (i.e., intact).

The cells can be human, mammalian, animal or plant cells.

According to a specific embodiment, the cells in the 3D structure are viable cells (e.g., more than 90%, 95%, or about 100% of the cells are viable).

Methods of determining cell viability are well known in the art and include but are not limited to Calcein AM, Clonogenic assay, Ethidium homodimer assay, Evans blue, Fluorescein diacetate hydrolysis/Propidium iodide staining (FDA/PI staining), Flow cytometry, Formazan-based assays (MTT/XTT), Green fluorescent protein, Lactate dehydrogenase (LDH), Methyl violet, Neutral red uptake (vital stain), Propidium iodide, DNA stain that can differentiate necrotic, apoptotic and normal cells, Resazurin, TUNEL assay.

According to a specific embodiment, the cells comprise a cell line.

According to a specific embodiment, the cells comprise immortalized cells.

According to a specific embodiment, the cells comprise healthy cells, or the majority of the cells in the 3D structure are healthy.

According to a specific embodiment, the cells comprise pathogenic cells, or the majority of the cells in the 3D structure are pathogenic.

According to a specific embodiment, the cells are of a single type.

According to a specific embodiment, the cells are of a number of types such as for example, stromal cells and tumor cells; endothelial cells and specialized cells and optionally connective tissue cells. For instance, vasculature cells and hepatocytes or cardiomyocytes.

According to a specific embodiment, the 3D structure is not subjected to fixing prior to injecting the dye.

According to a specific embodiment, the 3D structure is used fresh upon retrieval (e.g., up to 2 hours, 3 hours, 4 hours, 6 hours, 12 hours, 16 hours, 24 hours or 48 hours following retrieval).

Measures are taken to maintain cell viability e.g., placement in 4° C. and/or physiological buffer.

The present teachings also contemplate the use of 3D structures that have undergone freezing or cryopreservation as long as they can be recovered.

The 3D structure of cells may be used naïve or following or concurrent with a treatment such as testing the effect of a drug.

The 3D structure of cells can be of the cells alone or the cells can be embedded in a matrix, e.g., hydrogel.

Methods of embedding without fixing are well known in the art. Some are listed hereinbelow in the Examples section which follows (Example 1).

Various matrices can be used including but not limited to agar, gelatin, collagen, laminin and various hydrogels, or more complex compositions e.g., Matrigel®, which generally mimic an ECM environment.

Matrigel® is the trade name for a gelatinous protein mixture secreted by Engelbreth-Holm-Swarm mouse sarcoma cells and is a form of basement membrane extract. Matrigel® consists of several common ECM proteins including laminin, collagen and entactin, as well as various growth factors. It has previously been used to support a range of cell types in 3D culture (Amatangelo et al. 2013 Cell Cycle 12, 2113-2119; Lance et al. 2013 J Tissue Eng Regen Med. doi: 10.1002/term.1675; Li et al. 2013 Cell Tissue Res 354, 897-90).

Thus, once the 3D structure comprising the cells is at hand, at least one dye is injected into the structure to form an identifiable color pattern in said plurality of cells.

As used herein “a color pattern” refers to a staining resolution which is complex enough to index single cells or discrete cell populations in the 3D structure to a spatial position in the 3D structure.

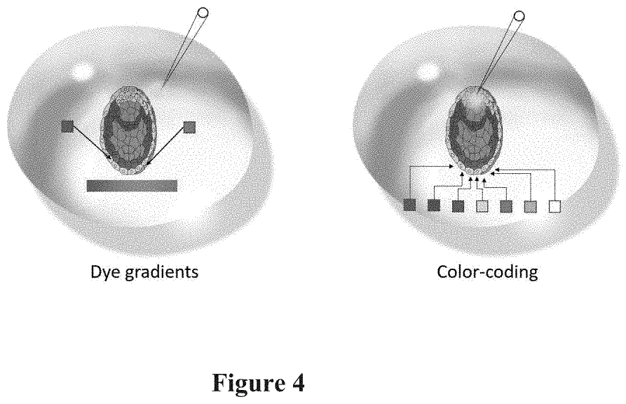

According to one embodiment, the injection comprises a plurality of injections and/or stains which form a color code of discrete cells or cell populations, as exemplified in FIG. 4, right panel.

According to another embodiment, the injection or injections generate a staining diffusion gradient, as exemplified in FIG. 4, left panel.

Hence, at least one dye can be introduced into the cell structure or into the matrix embedding the cells. The latter is especially useful when relying on a staining diffusion gradient.

As used herein “staining diffusion gradient” refers to a color pattern which is achieved by way of diffusion in the 3D structure.

According to a specific embodiment, the color pattern comprises a diffusion gradient. A diffusion gradient can be in the form of a gradually changing intensity of a color over the 3D structure, wherein the gradual change in the intensity results from diffusion of the respective dye from the location of its injection into the 3D structure.

According to a specific embodiment, the color pattern comprises a plurality of colors, each characterized by a different hue or central wavelength, also referred to as color code. In these embodiments, the color pattern can form a map of colors, wherein each region on the map is chartered by a different color (e.g., expressed using coordinates over the CIE L*a*b*space) or a different central wavelength.

The present embodiments also contemplate color patterns which are combinations of diffusion gradients and a plurality of colors. In these embodiments the color pattern is in the form of a gradually changing intensity for each of a plurality of colors, each characterized by a different hue or central wavelength.

Regardless if the pattern is achieved by a color code or a staining diffusion pattern, the staining pattern can be achieved by a single injection and single dye (relevant for diffusion), multiple injections, different positions, multiple dyes (e.g., 2, 3, 4, 5, 6, 7, and more), multiple concentrations of a single or multiple dyes and combinations of same.

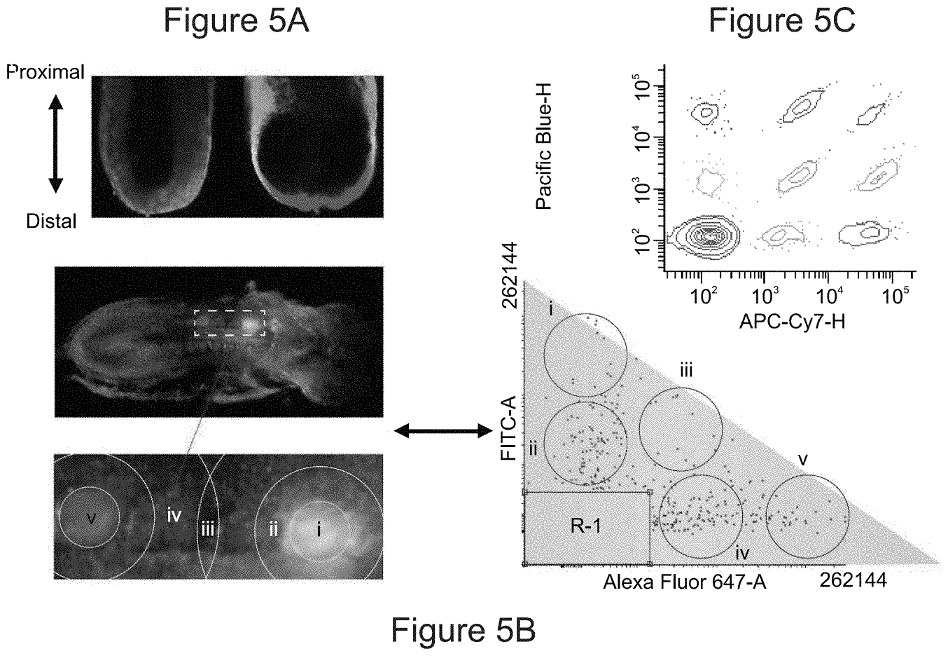

Thus, as mentioned a plurality of dyes and/or a plurality of concentrations are used in order to obtain a sufficiently complex pattern. For example, as shown in FIG. 5C—using a simple coloring scheme with two colors and three distinctly separated dye concentrations, nine color codes were generated (FIG. 5C). This approach enables pooling cells from different embryos, and following index sorting, un-mix them during subsequent analysis.

The selection of dyes naturally depends on the type of pattern desired. Some features are shared by both types of staining while others are specific e.g., for staining diffusion gradient.

Thus, according to a specific embodiment, the dye is at least one of:

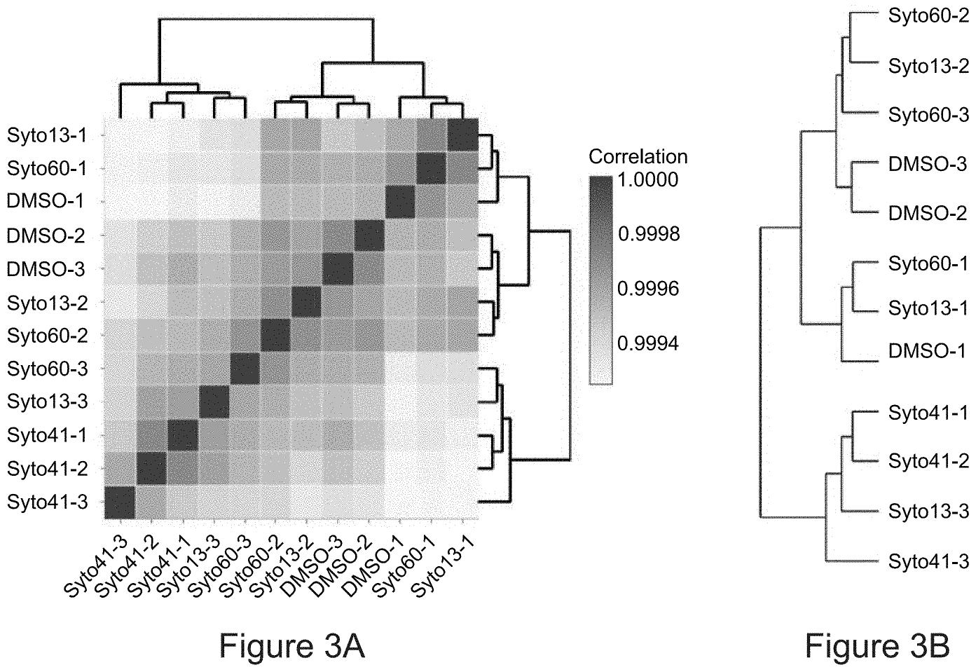

(i) non-toxic-as mentioned, the cells are kept viable in order not to affect the single cell analysis to be followed. One potential obstacle with cellular dyes, in general, is toxicity or other adverse side-effects. To ensure the dyes do not affect transcription, a toxicity assay can be carried out on a specific cell population and their composition analyzed such as by transcriptome analysis through RNAseq. The cells are incubated for a predetermined time period with the tested dye to mimic in-vivo conditions, washed and processed for RNAseq. When the analysis reveals no significant differences between the samples, the dye is considered non-toxic as staining does not affect transcription at the time scales relevant to the methodology (see for instance FIGS. 3A-B).

(ii) membrane permeable—to allow diffusion into cells, retention in cells and through tissues;

(iii) capable of forming staining diffusion gradient—Thus, according to an embodiment, the dyes are screened for forming a diffusion gradient in the 3D structure of cells. Following is an exemplary embodiment: to validate dye gradient formation, one of the dyes is injected into the gel adjacent to the distal pole of an E7.5 embryo and the formation of a staining gradient is validated. To assess the interaction between two dyes, the two dyes are applied to the proximal and distal poles of the embryo, respectively and the formation of opposing and coalescing gradients is observed (as exemplified in FIG. 5A). Next, the diffusion and gradient formation are assessed on larger scales in E8.5 embryos, by injecting the two dyes directly into adjacent somites (as exemplified in FIG. 5B). FACS analysis of somite cells following enzymatic dissociation revealed that intensely stained cells exhibit a singular staining pattern, while cells labeled by both dyes have lower signal intensities. This validated the practicality of using dyes for indexing spatial position, as well as dye retention in-vivo; and

(iv) not leaking from cells following said isolating—they must not leak from cells in suspension, thus skewing the analysis.

Samples stained with leaky dyes are easily identifiable as the distributions of their signal intensity peaks are shifted in the pooled samples compared with the single-concentration samples. Hence dyes that exhibit mild to massive cross-staining between samples are rejected.

According to a specific embodiment, the dye is a nucleic acid binding dye.

According to a specific embodiment, the dye is a fluorescent dye.

Non-limiting examples of fluorescent dyes include SYBR green; SYBR blue; DAPI; propidium iodine; Hoeste; SYBR gold; ethidium bromide; acridines; proflavine; acridine orange; acriflavine; fluorcoumanin; ellipticine; daunomycin; chloroquine; distamycin D; chromomycin; homidium; mithramycin; ruthenium polypyridyls; anthramycin; phenanthridines and acridines; propidium iodide; hexidium iodide; dihydroethidium; ethidium monoazide; ACMA; Hoechst 33258; Hoechst 33342; Hoechst 34580; DAPI; acridine orange; 7-AAD; actinomycin D; LDS751; hydroxystilbamidine; SYTOX Blue; SYTOX Green; SYTOX Orange; POPO-1; POPO-3; YOYO-1; YOYO-3; TOTO-1; TOTO-3; JOJO-1; LOLO-1; BOBO-1; BOBO-3; PO-PRO-1; PO-PRO-3; BO-PRO-1; BO-PRO-3; TO-PRO-1; TO-PRO-3; TO-PRO-5; JO-PRO-1; LO-PRO-1; YO-PRO-1; YO-PRO-3; PicoGreen; OliGreen; RiboGreen; SYBR Gold; SYBR Green I; SYBR Green II; SYBR DX; SYTO-40, SYTO-41, SYTO-42, SYTO-43, SYTO-44, and SYTO-45 (blue); SYTO-13, SYTO-16, SYTO-24, SYTO-21, SYTO-23, SYTO-12, SYTO-11, SYTO-20, SYTO-22, SYTO-15, SYTO-14, and SYTO-25 (green); SYTO-81, SYTO-80, SYTO-82, SYTO-83, SYTO-84, and SYTO-85 (orange); SYTO-64, SYTO-17, SYTO-59, SYTO-61, SYTO-62, SYTO-60, and SYTO-63 (red); fluorescein; fluorescein isothiocyanate (FITC); tetramethyl rhodamine isothiocyanate (TRITC); rhodamine; tetramethyl rhodamine; R-phycoerythrin; Cy-2; Cy-3; Cy-3.5; Cy-5; Cy5.5; Cy-7; Texas Red; Phar-Red; allophycocyanin (APC); Sybr Green I; Sybr Green II; Sybr Gold; CellTracker Green; 7-AAD; ethidium homodimer I; ethidium homodimer II; ethidium homodimer III; umbelliferone; eosin; green fluorescent protein; erythrosin; coumarin; methyl coumarin; pyrene; malachite green; stilbene; lucifer yellow; cascade blue; dichlorotriazinylamine fluorescein; dansyl chloride; fluorescent lanthanide complexes such as those including europium and terbium; carboxy tetrachloro fluorescein; 5 and/or 6-carboxy fluorescein (FAM); 5- (or 6-) iodoacetamidofluorescein; 5-{[2(and 3)-5-(Acetylmercapto)-succinyl]amino} fluorescein (SAMSA-fluorescein); lissamine rhodamine B sulfonyl chloride; 5 and/or 6 carboxy rhodamine (ROX); 7-amino-methyl-coumarin; 7-Amino-4-methylcoumarin-3-acetic acid (AMCA); BODIPY fluorophores; 8-methoxypyrene-1;3;6-trisulfonic acid trisodium salt; 3;6-Disulfonate-4-amino-naphthalimide; phycobiliproteins; AlexaFluor 350, 405, 430, 488, 532, 546, 555, 568, 594, 610, 633, 635, 647, 660, 680, 700, 750, and 790 dyes; DyLight 350, 405, 488, 550, 594, 633, 650, 680, 755, and 800 dyes; and other fluorophores.

The dye may comprise an organometallic fluorophore. Non-limiting examples of organometallic fluorophores include lanthanide ion chelates, non-limiting examples of which include tris(dibenzoylmethane) mono(1,10-phenanthroline)europium(III), tris(dibenzoylmethane) mono(5-amino-1,10-phenanthroline)europium (III), and Lumi4-Tb cryptate.

According to a specific embodiment, the dye does not have a binding specificity to a specific gene/RNA.

According to a specific embodiment, the dye is a cell permeant cyanine dye.

According to a specific embodiment, the cell permeant cyanine dye is a SYTO dye (available from ThermoFisher).

A SYTO dye can stain both RNA and DNA.

According to a specific embodiment, the dye is selected from the group consisting of Syto13, Syto41, Syto60 and a combination of same.

Following a predetermined time in which the stain is allowed to diffuse in the 3D structure of cells, the structure is subjected to imaging. The imaging is preferably by microscopy imaging, optionally and preferably high-resolution microscopy imaging, and can be by a single capture, or by scanning, e.g., by means of confocal microscopy. The imaging is preferably by a pixelated image sensor that is sensitive to the characteristic fluorescence emission wavelength(s) of the dye(s).

According to a specific embodiment, identifying the color pattern in the 3D structure is effected by imaging, e.g., fluorescent signal collection.

Once an image of the 3D structure is obtained, it is optionally and preferably transmitted to an image processor configured for receiving the image and executing the operations described below, by executing computer program having a plurality of program instructions.

Computer programs implementing the method of the present embodiments can commonly be distributed to users on a distribution medium such as, but not limited to, a floppy disk, a CD-ROM, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The image processing technique of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

The image to be analyzed using the teachings of the present embodiments is generally in the form of imagery data arranged grid-wise in a plurality of picture-elements (e.g., pixels, group of pixels, etc.).

The term “pixel” is sometimes abbreviated herein to indicate a picture-element. However, this is not intended to limit the meaning of the term “picture-element” which refers to a unit of the composition of an image.

References to an “image” herein are, inter alia, references to values at picture-elements treated collectively as an array. Thus, the term “image” as used herein also encompasses a mathematical object which does not necessarily correspond to a physical object. The original and processed images certainly do correspond to physical objects which are the 3D structures from which the imaging data are acquired.

Each pixel in the image can be associated with a single digital intensity value, in which case the image is a grayscale image. Alternatively, each pixel is associated with three or more digital intensity values sampling the amount of light at three or more different color channels (e.g., red, green and blue) in which case the image is a color image. Also contemplated are images in which each pixel is associated with a mantissa for each color channels and a common exponent (e.g., the so-called RGBE format). Such images are known as “high dynamic range” images.

In some embodiments of the present invention the image is processed to identify the color pattern formed by the dye(s). This is optionally and preferably performed by binning the image into a plurality of spatial bins. Each spatial bin is interchangeably referred to herein as an “sbin.”

A spatial bin is a region of the image and is therefore a collection of picture-element (e.g. a collection of pixels). The spatial bin is typically, but not necessarily, a continuous region over the image. The region is “continuous” in the sense that each of the picture-elements that belong to a particular spatial bin is adjacent to one or more other picture-elements of the same spatial bin. In other words, when the spatial bin is a continuous region over the image, there are no isolated picture-elements in it. In some embodiments of the present invention, at least one of the spatial bins, more preferably each of the spatial bins, forms a simply connected region over the image. When a particular spatial bin forms a simply connected region, it includes a collection of picture-elements in which any two picture-elements can be connected by a line (not necessarily a straight line) that passes only through picture-elements of the particular spatial bin, without intersecting picture-elements of other spatial bins.

In some embodiments of the present invention the binning is performed in a tissue-specific manner, namely, the bins are selected separately for each tissue under analysis, or in a predetermined manner, irrespectively of the tissue or the 3D structure. The advantage of the latter embodiments is that the 3D structure is discretized, while allowing a common reference onto which different specimens can be projected by markup and alignment over the image.

Once the spatial bins are defined (either in a predetermined manner, or in a tissue-specific manner) the relative abundance of cells in each spatial bin is optionally and preferably estimated. This can be done in more than one way. In some embodiments of the present invention relative abundance is estimated based on confocal microscopy data. In these embodiments a confocal microscope can be used to create a 3D model of the cell positions, and the number of cells that are projected into each of the spatial bins can be counted, thereby estimating the frequencies. When no confocal microscopy data are available, the frequencies can be inferred from the image data itself, and/or based on the structure of the tissue. For example, such inference can be obtained for tissues for which known spatial features have estimated typical volumes, and for which volumes of each cell types can be assumed based on past measurements.

In some embodiments of the present invention the color pattern is identified by thresholding the picture-elements in the image, so as to binary classify each picture-element as being either stained or non-stained. Thus, when dyes of n colors are used, thresholding can be applied to the cell fluorescence, so as to define 2ⁿ possible fluorescence states for each picture-element cell. The thresholding can be according to any known technique, such as, but not limited to, Otsu's method, use of a fixed and predetermined set of threshold values, or some other clustering procedure, e.g., K-means.

In some embodiments of the present invention the thresholding is based on an estimated number of cells per staining characteristic in the image. For example, the fraction of cells having each fluorescent state (e.g., each of the 2ⁿ states, in the above example) can be estimated as the fraction of picture-elements having that state out of the total number of picture-element in the spatial bin. This fraction can optionally and preferably be combined with the fraction of total cells within each spatial bin (the ratio between the number of cells in a particular sbin and the number of cells in all the sbins) so as to estimate, for each tissue, the expected number of cells for each color (each of the n colors, in the above example). Then a threshold can be selected, for each measured color intensity, so as to ensure that the expected number of cells is considered as having the relevant channel “on”.

The Inventors found that the identified color pattern can be used as an index mechanism that maps between spatial position and staining characteristic (hue, wavelength, intensity, etc.).

The present embodiments therefore contemplate using the identified color pattern for identifying the spatial position of a single cell having a known staining characteristic. In these embodiments, the staining characteristic of a single cell or a group of single cells is obtained from an external source. For example, the staining characteristic can be read from a computer readable medium storing staining characteristics of single cells. Such staining characteristics can be obtained by applying to cells isolated from the 3D structure one or more assays selected from the group consisting of fluorescence microscopy, confocal microscopy, fluorescence automated plate reading, flow cytometry, FACS) assay or confocal microscopy. The obtained staining characteristic can then be aligned to the index that so as to identify the spatial position of the single cell(s).

In some embodiments of the present invention the aligning comprises estimating likelihood for a plurality of cell types to have a respective plurality of staining characteristics. Such likelihood can be expressed as a sum of terms, each describing a probability for the particular cell type to be in a particular spatial bin. The estimated likelihood can optionally be optimized by an optimization procedure, which is preferably a non-linear optimization procedure. Representative examples of optimization procedures suitable for the present embodiments include, without limitation, a steepest descent procedure, conjugate-gradients procedure, and a quasi-Newton procedure. In some embodiments of the present invention the optimization procedure comprises a Broyden-Fletcher-Goldfarb-Shanno (BFGS) procedure, preferably an L-BFGS procedure, and in some embodiments of the present invention the optimization procedure comprises Monte-Carlo simulation.

As stated, the cells can be isolated step from the structure.

Measures are taken not to affect the staining of the cell even after isolation so it's staining can be analyzed by any known method such as by fluorescence activated cell sorter (FACS).

According to a specific embodiment, the dissociation step complies with further steps of cell analysis e.g., transcriptome. Inadequate methods for tissue dissociation generate considerable loss in the quantity of single cells produced and in the produced cells' viability. Improper dissociation may also demote the quality of data attained in functional and molecular assays due to the presence of large quantities cellular debris containing immune-activatory danger associated molecular patterns, and due to the increased quantities of degraded proteins and RNA.

Methods of isolating single cells include, but are not limited to micromanipulation, laser capture microdissection, microfluidics, manual picking, enzymatic digestion, and Raman tweezers.

According to a specific embodiment, the isolation is done by enzymatic dissociation such as by using rypsin A, Collagenase, Papain, or a combination of same e.g., a commercial enzyme cocktail (e.g. Myltenyi multi-tissue dissociation kit).

Regardless of the method employed, the single cells are imaged such as by FACS or confocal microscopy and their staining determined.

The cells are then subjected to single cell analysis.

Separating of various types of molecules from a single cell is the point of initiation of measurement for single-cell analysis of DAN, RNA, protein (peptides), metabolites, epigenetics, carbohydrates, lipids and more.

According to a specific embodiment, a technology used for genomics, epigenomics and transcriptomics include DNA sequencing and microarrays (planar, bead, and fiber-optic arrays). For proteomics, mass spectrometry (MS) and protein arrays and metabolomics, MS and NMR. All these can be collectively referred to as “Omics”. These can be done in an automated and/or miniaturized manner.

WO2014/108850 describes various methods for single cell transcriptomics. Wang and Bodovits 2010 Trends Biotechnol. 28(6):281-290 teach methods for single cell Omics.

Following are some available and non-limiting examples of single cell nucleic acid analysis and epigenomics.

TABLE 1

| Type of -Seq** | Description | Analysis Tools** |
|---|---|---|
| RNA-Seq | Identify expression levels of | STAR, kallisto, salmon, |
| | genes/isoforms, gene fusion, | RSEM, HISAT2, |
| | mutations/SNPs, alternative | HTSeq-count, subreads |
| | gene spliced transcripts, and | featureCount, DESeq2, |
| | more. | edgeR, |
| | | Stringtie/Ballgown |
| Whole genome/ | Identify SNPs, INDELs and | BWA, Bowtie2, GATK, |
| exome | structural variants | Freebayes, Lumpy |
| ChIP-Seq | Identify the binding sites of | Bowtie2, MACS2, |
| | DNA-associated proteins | Homer |
| ATAC-Seq | Identify chromatin accessibility | Bowtie2, MACS2, |
| | | Homer |
| Methylation | Identify hyper/hypo-methylated | Bismark, methylKit, |
| | regions of the DNA (CpG | bsseq, bis-snp |
| | Islands, shores, shelves, | |
| | etc . . . ) | |
| microRNA-Seq | Identify microRNAs that | CAP-miRSeq, |
| | modulate protein expression | miARma-Seq |
| | through transcript degradation, | |
| | inhibition of translation, or | |
| | sequestering transcripts | |
| Single-cell | Examine the sequence | STAR, kallisto, salmon, |
| RNA-Seq^{#} | information from individual | scater, Seurat |
| | cells providing a higher | |
| | resolution of cellular | |
| | differences and a better | |
| | understanding of the function | |
| | of an individual cell in the | |
| | context of its | |
| | microenvironment | |

In principle Omics analysis typically relies on unique molecular labeling of the cells which are being analyzed (e.g., by the use of barcodes) but in this case the cell is also characterized by its location in the 3D structure. The acquired data is stored in a certain manner, for example, in specific data structure(s), for consumption by one or more processors (or processing cores) that are configured to access the data structures and to perform computational analysis such that biologically meaningful patterns within the 3D structure are detected. The computational analysis and associated computer-generated visualization of results of the computational analysis on a graphical user interface allow for the observation of properties of the 3D structure that would not otherwise be detectable. In particular, in some embodiments, each cell of the 3D structure is subjected to analysis and characteristics of each cell within the sample are obtained such that it becomes possible to characterize the 3D structure based on differentiation among different types of cells in the 3D structure. For example, data analysis can reveal distributions of cell

Thus, the present teachings, in some embodiments thereof relate to a method of deriving spatially reconstruction of a tissue of interest as follows:

1. Using initial experiments and prior knowledge, a working model for the spatial organization in the tissue of interest is developed. This involves any number of annotated spatial regions and a scheme defining their relative 2D or 3D localization.
2. Tissues are obtained for initial staining. Several specimens from the same tissue (in models showing reproducible tissue morphology), or alternatively sections of single tissues are studied under the microscope. Alignment between the annotated spatial model (1) and the specimens is determined, and foci for dye injection are selected based on the study aims (typically focusing on specific spatial regions in the model).
3. Images of diffused dyes are acquired prior to cell disassociation. Using dedicated software, images are annotated to define the location of the regions in the proposed spatial model. The distribution of pixel intensity within each region is estimated and saved for further processing.
4. Cells are disassociated and sorted by FACS while recording fluorescence levels in all dyes' channels. scRNA-seq is performed using MARS-seq (alternatively, any single cell strategy can be performed).
5. Single cell RNA-seq profiles or any other profile with attached dye intensity levels are organized in a database. By combing these with the intensity distributions per region as determined in stage 3, it is possible to infer for each transcriptional state (defined by a collection of single cells, or a Metacell) a model for its spatial distribution (probability for observation in each of the spatial regions defined in stage 1).
6. Based on the results, spatial regions can be redefined to facilitate inference of refined or more targeted spatial structure.

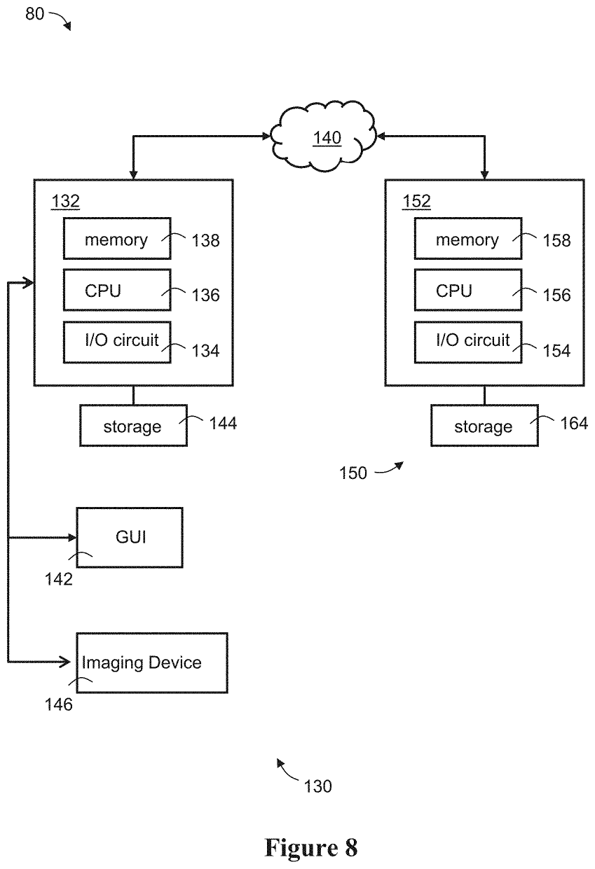

Reference is now made to FIG. 8, which is a schematic illustration of a system 80 for identifying a position of a single cell in an image of cells, according to some embodiments of the present invention. System 80 can comprise a client computer 130 having a hardware processor 132, which typically comprises an input/output (I/O) circuit 134, a hardware central processing unit (CPU) 136 (e.g., a hardware microprocessor), and a hardware memory 138 which typically includes both volatile memory and non-volatile memory. CPU 136 is in communication with I/O circuit 134 and memory 138. Client computer 130 preferably comprises a graphical user interface (GUI) 142 in communication with processor 132. I/O circuit 134 preferably communicates information in appropriately structured form to and from GUI 142.

In some embodiments of the present invention system 80 also comprises a server computer 150 which can similarly include a hardware processor 152, an I/O circuit 154, a hardware CPU 156, a hardware memory 158. When system 80 comprises a client and a server computers, the I/O circuits 134 and 154 of client 130 and server 150 computers can operate as transceivers that communicate information with each other via a wired or wireless communication. For example, client 130 and server 150 computers can communicate via a network 140, such as a local area network (LAN), a wide area network (WAN) or the Internet. Server computer 150 can be in some embodiments be a part of a cloud computing resource of a cloud computing facility in communication with client computer 130 over the network 140.

In some embodiments of the present invention system 80 comprises an imaging device 146 that is associated with client computer 130, and that is capable of imaging a 3D structure containing cells. For example, imaging device 146 that can be microscopy imaging device, configures for acquiring a microscopy image of the 3D structure by a single capture, or by scanning.

GUI 142 and processor 132 can be integrated together within the same housing or they can be separate units communicating with each other. GUI 142 can optionally and preferably be part of a system including a dedicated CPU and I/O circuits (not shown) to allow GUI 142 to communicate with processor 132. Processor 132 issues to GUI 142 graphical and textual output generated by CPU 136. Processor 132 also receives from GUI 142 signals pertaining to control commands generated by GUI 142 in response to user input. GUI 142 can be of any type known in the art, such as, but not limited to, a keyboard and a display, a touch screen, and the like. In preferred embodiments, GUI 142 is a GUI of a mobile device such as a smartphone, a tablet, a smartwatch and the like. When GUI 142 is a GUI of a mobile device, processor 132, the CPU circuit of the mobile device can serve as processor 132 and can execute the code instructions described herein.

Client 130 computer and server 150 computer (when employed) can further comprise one or more computer-readable storage media 144, 164, respectively. Media 144 and 164 are preferably non-transitory storage media storing computer code instructions for executing the image processing technique described herein, and processor 132 and/or processor 152 access the storage media and execute these code instructions. The code instructions can be run by loading the respective code instructions into the respective execution memories 138 and 158 of the respective processors 132 and 152.

Each of storage media 144 and 164 can store program instructions which, when read by the respective processor, cause the processor to receive an image of the cells, and a staining characteristic of a single cell, and to execute the image processing technique described herein. In some embodiments of the present invention, an input image containing the cells is generated by imaging device 130 and is transmitted to processor 132 by means of I/O circuit 134. Processor 132 can receive the staining characteristic of a single cell from GUI 142 or from storage medium 144, and the image of the cells from imaging device 130, determine the position of the single cell and generate on GUI 142 an output pertaining to the determined position. Alternatively, processor 132 can transmit the image of the cells and the staining characteristic of the single cell over network 140 to server computer 150. Computer 150 receives the image and the staining characteristic, determines the position of the single cell, as further detailed hereinabove, and transmits the determined position back to computer 130 over network 140. Computer 130 receives the determined position and generate on GUI 142 an output pertaining to the determined position.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Optimization of Stable Single Cell Fluorescence Labeling

Special care had to be employed in the choice of fluorescent dyes. The selected dyes must be: (i) non-toxic at the time-scales relevant to our experiments (to avoid having an effect on the cells' transcriptional profile). (ii) Membrane permeable to allow diffusion into cells and through tissues. (iii) Generate concentration-dependent staining at relevant concentrations, and (iv) be retained in the cells upon enzymatic dissociation into single-cell suspensions. Likewise, they must not leak from cells in suspension, thus skewing the analysis. After careful consideration and testing, a set of nucleic-acid binding dyes called Syto dyes (Invitrogen) was selected. Each dye in the set has differential nucleic-acid binding properties, requiring individual characterization, and they can be purchased as three separate kits (Syto Red, Green and Blue), allowing combinatorial staining.

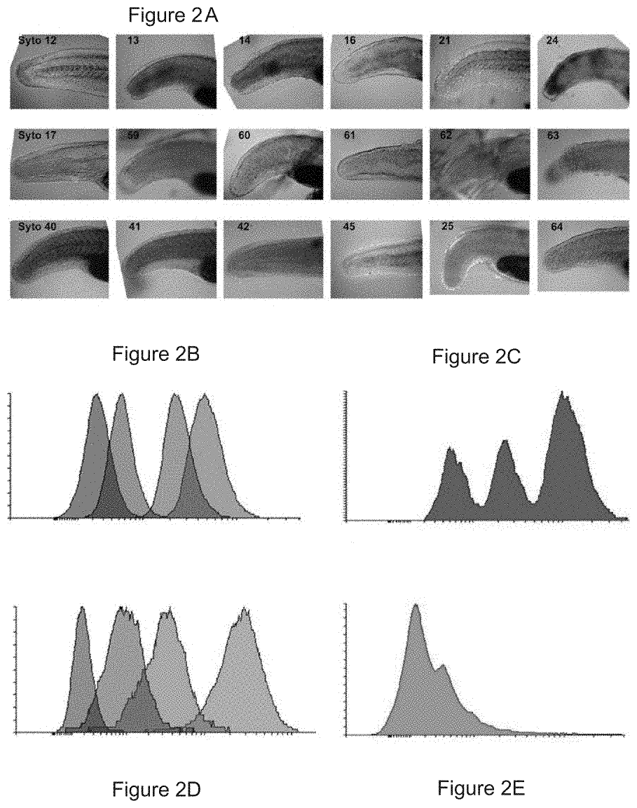

As a preliminary characterization step, the zebrafish larvae (48 hours post-fertilization) were selected since they can easily be obtained in large numbers. The larvae were immobilized using 4% Tricane and embedded in Matrigel droplets which were then allowed to gel at 37° C. for 15 minutes. Next, a droplet of each Syto dye was injected adjacent to the tail and allowed the dye to diffuse into the live tissue for 15 minutes, followed by imaging (FIG. 2A). All 3 were injected to the gel. Dyes that exhibited a good penetration range into the tail tissue were screened for, thus precluding several short-range dyes. The dyes were further screened to preclude cross-contamination of the signal between cells (leakiness). For this, cohorts of suspended mouse embryonic stem cells (mESCs) were used. Each cohort of 1×10⁶ was divided into four samples and stained with log-fold concentrations of a single dye, ranging from unstained to 0.05 mM final concentrations (FIG. 2B). The cells were next pelleted, washed extensively with PBS and re-suspended.

Using a cell sorter device, the fluorescent signal from each sample was analyzed. Next, all four samples were pooled from each cohort and the signal re-analyzed in order to detect dye leakage and cross-staining between cells. Samples stained with leaky dyes were easily identifiable as the distributions of their signal intensity peaks were shifted in the pooled samples compared with the single-concentration samples. Hence an additional handful of dyes that exhibited mild to massive cross-staining between samples were rejected. Eventually a single dye from each Syto set was selected that had suitable properties: Syto13 (Green, emission 509-514 nm); Syto60 (Far red, emission 678 nm); Syto41 (Blue, emission 454).

One potential obstacle with cellular dyes, in general, is toxicity or other adverse side-effects. To ensure the dyes do not affect transcription, a toxicity assay was carried out on mouse embryonic stem cells and their transcriptome analyzed through RNAseq. Mouse ESCs suspensions were distributed into 12 vials. Nine vials (3 biological replicates) were stained with high concentrations of each dye (0.05 mM) and three served as non-treated control (corresponding dilution of DMSO). The cells were incubated for 1 hr with each dye to mimic in-vivo conditions, washed and processed for RNAseq. The analysis revealed no significant differences between the samples, demonstrating that dye staining does not affect transcription at the time scales relevant to our experiments (FIG. 3A-B).

### Example 2

### Generating Fluorescent Gradients and Color-Coding of Tissues

The selected Syto cohort can be used to generate fluorescent gradients in intact tissues as well as combinatorial color-coding, using the concentration-dependent fluorescent intensity property of the dyes (FIG. 4). To test the efficacy of the dyes in generating gradient in intact tissues, early mouse post-implantation embryos were used as a model. Embryos from different stages of development were embedded in matrigel droplets as illustrated in FIG. 4. To validate dye gradient formation, one of the dyes (Syto13) was injected into the matrigel adjacent to the distal pole of an E7.5 embryo and the formation of a staining gradient was validated. To assess the interaction between two dyes, Syto13 and 60 were applied to the proximal and distal poles of the embryo, respectively and the formation of opposing and coalescing gradients was observed (FIG. 5A). Next, the diffusion and gradient formation were assessed on larger scales in E8.5 embryos, by injecting Syto13 and Syto60 directly into adjacent somites (FIG. 5B). FACS analysis of somite cells following enzymatic dissociation revealed that intensely stained cells exhibit a singular staining pattern, while cells labeled by both dyes have lower signal intensities. This validated the practicality of using the dyes for indexing spatial position, as well as dye retention in-vivo.

One major challenge facing sorting-dependent single cell sequencing of rare cell populations is the minimal quantities of available biological material. To overcome this challenge, it is often necessary to pool several samples for each cohort. However, especially for systems that exhibit rapid dynamics, this may come at a considerable cost of reducing the resolution and complexity. Furthermore, in clinical samples, this problem becomes even more acute as there is often no comparable material to pool together. To address this, the present inventors sought to take advantage of the property of the dyes to generate a concentration-dependent fluorescent signal, in single cells (see FIG. 2C-D). Thus, individual E6.0 embryos (containing ˜400 cells) were dissociated to single cells, followed by color coding based on different dye concentrations. To this end, using a simple coloring scheme with two colors and three distinctly separated dye concentrations, nine color codes were generated (FIG. 5C). This approach enables pooling cells from different embryos, and following index sorting, un-mix them during subsequent analysis.

### Example 3

### The StainSeq Pipeline

Given the development of a strategy for stable fluorescent indexing of single cells, the StainSeq pipeline was designed for deriving spatially reconstruction of a tissue of interest as follows:

1. Using initial experiments and prior knowledge, a working model for the spatial organization in the tissue of interest is developed. This involves any number of annotated spatial regions and a scheme defining their relative 2D or 3D localization.
2. Tissues are obtained for initial staining. Several specimens from the same tissue (in models showing reproducible tissue morphology), or alternatively sections of single tissues are studied under the microscope. Alignment between the annotated spatial model (1) and the specimens are determined, and foci for dye injection are selected based on the study aims (typically focusing on specific spatial regions in the model).
3. Images of diffused dyes are acquired prior to cell disassociation. Using dedicated software, images are annotated to define the location of the regions in the proposed spatial model. The distribution of pixel intensity within each region is estimated and saved for further processing.
4. Cells are disassociated and sorted by FACS into 384 well plates while recording fluorescence levels in all dyes' channels. scRNA-seq is performed using MARS-seq (alternatively, any single cell strategy can be performed).
5. Single cell RNA-seq profiles with attached dye intensity levels are organized in a database. By combing these with the intensity distributions per region as determined in stage 3, we can infer for each transcriptional state (defined by a collection of single cells, or a Metacell) a model for its spatial distribution (probability for observation in each of the spatial regions defined in stage 1).
6. Based on the results, spatial regions can be redefined to facilitate inference of refined or more targeted spatial structure.

### Example 4

### StainSeq Computational Modelling and Inference

1. Definitions of Sbins and Estimation of their Frequencies

The model was initiated by portioning a model of interest into idealized spatial bins, referred herein as sbins. Each sbin encapsulated a defined volume in the tissue, which can be internally complex. The collection of sbins that is defined discretizes the spatial structure, while allowing a common reference onto which different specimens can be projected by markup and alignment of the sbin regions over a high-resolution microscopy image.

No implicit assumptions on the number of cells encapsulated within each sbin were made. Instead, the model assumed some probability distribution P(s) to define the relative abundance of cells in the s-th sbin. In experiments on tissues such as the mouse embryo, a confocal microscope can be used to create a 3D model of the cell positions, and the number of cells that are projected into each of the sbins can be counted, thereby estimate the probabilities P(s). In other scenarios, these probabilities are optionally and preferably inferred from the data and/or from prior assumptions on the structure of the tissue.

### 2. Modeling the Cells' Fluorescence

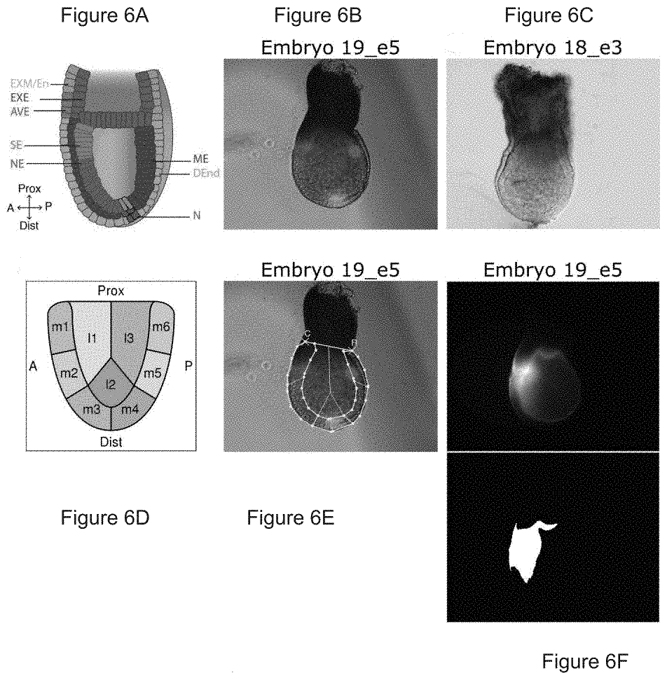

In this Example, dyes of three colors were used. Cell fluorescence in each channel was treated as a binary variable with only two possible values: “on” and “off”. Thus, there were 23=8 possible fluorescence states for a cell. For each sample, the fraction of cells having each fluorescence within each of the sbins was estimated by first manually annotating the edges of the sbin over each tissue's image (see, e.g., FIG. 6E). Otsu's method was then applied to the complete image of each fluorescent channel so as to mark each pixel as being either fluorescent or not fluorescent (see, e.g., FIG. 6F). In this Example, the relation between the number of pixels and the number of cells within each sbin was assumed to be linear. Thus, the fraction of cells having each fluorescent state was estimated as the fraction of pixels having that state out of the total number of pixels in the sbin. However, this need not necessarily be the case, and non-linear relation between the number of pixels and the number of cells is also contemplated.

The fraction of cells with fluorescent state f within sbin s of assayed tissue e is denoted P(f|s, e).

The estimated fraction was combined with the distribution of total cells over all the sbins so as to estimate, for each tissue, the expected number of cells for each color (green, blue or red, in the present Example). For each tissue, a threshold was selected for each color intensity that was measured by the FACS. The thresholds (one thresholds per tissue per color, in the present Example were selected to ensure that the expected number of cells is considered as having the relevant channel “on”.

### 3. Inferring Positions of Cell Types

For simplicity, scRNA-seq data was used only to determine one label per cell, where the label can be interpreted initially as the cell type. In this Example, it was assumed that there exists a single joint distribution over cell types and sbins from which the cells of all analyzed tissues were sampled. The probability of a sampled cell to be of cell type c and reside within sbin s is denoted P(s, c).

The result of the experiment described in this Example is a series of observed cell types cᵢ and fluorescent states fᵢ, coming from tissues eᵢ. The log likelihood of the observed series is given by:

$$log L = log {\prod }_{i} P ( {c}_{i} , {f}_{i} \vert {e}_{i} ) = {\sum }_{i} log P ( {c}_{i} , {f}_{i} \vert {e}_{i} )$$

Looking at one term of this sum, one has:

$$\begin{matrix}P ( {c}_{i} , {f}_{i} \vert {e}_{i} ) = P ( {c}_{i} \vert {e}_{i} ) \cdot P ( {f}_{i} \vert {c}_{i} , {e}_{i} ) \\ = P ( {c}_{i} \vert {e}_{i} ) \cdot {\sum }_{s} P ( {f}_{i} \vert s , {c}_{i} , {e}_{i} ) \cdot P ( s \vert {c}_{i} , {e}_{i} ) \\ = P ( {c}_{i} \vert {e}_{i} ) \cdot {\sum }_{s} P ( {f}_{i} \vert s , {c}_{i} , {e}_{i} ) \cdot \frac{P ( s , {c}_{i} \vert {e}_{i} )}{P ( {c}_{i} \vert {e}_{i} )} \\ = {\sum }_{s} P ( {f}_{i} \vert s , {c}_{i} , {e}_{i} ) \cdot P ( s , {c}_{i} \vert {e}_{i} )\end{matrix}$$

$$P ( {e}_{i} ) = P ( {e}_{i} ) \cdot P ( {c}_{i} , {e}_{i} ) = P ( {e}_{i} ) \cdot {\sum }_{s} P ( s , {c}_{i} , {e}_{i} ) \cdot P ( {c}_{i} , {e}_{i} )$$

To simplify the last summation terms we used independence assumptions.

For the first multiplicative term in each summand, it is assumed that the distribution of fluorescent states is independent of the cell type cᵢ given s and eᵢ. Similarly, it is assumed that the joint distribution of sbin and cell type is independent of the assayed tissue, therefore:

$$- log L = - {\sum }_{i} log [ {\sum }_{s} P ( {f}_{i} \vert s , {e}_{i} ) \cdot P ( s , {c}_{i} ) ]$$

The term P(fᵢ|s, eᵢ) on the right-hand side was already calculated from the tissues' images. A maximum likelihood estimate for the joint distribution P(s,cᵢ) can be found, for example, by minimizing −log L. In the present Example, this minimization was performed using the L-BFGS algorithm, implemented by the scipy.optimize.fmin_1_bfgs_b( ) function (from the scipy python package) and with random initialization.

The inferred distribution need not necessarily be the expected marginal P(s) that was measured using the confocal microscope. The inferred joint distribution is therefore denoted as P′(s). To enforce the constraint P′(s)=P(s) the Kullback-Leibler divergence between P′(s) and P(s) was added to the minimized function:

f=−log L+λ·D_{KL}[P′(s)|IP(s)]

The parameter λ was iteratively and exponentially increased, starting with zero and using the solution of each iteration as the initialization of the next iteration. This process was continued until the Kullback-Leibler divergence becomes less than a divergence threshold. The divergence threshold can be determined in advance for all types of tissue, or, more preferably selected based on the data and optionally and preferably also based on the number of sbins.

### Example 5

### Application to Mouse Embryos

The new system was used to assess the post-implantation embryo (gestational age E7.5). At this developmental stage, the embryo already includes a large array of distinct cell types with well-defined localization. In addition, the cells of the E7.5 embryo are still composed of three distinct layers of cells, corresponding to the three germ layers (FIG. 6A). This allows to localize cells to the right layer based on their transcriptional profile alone.

Embryos were dissected at the correct gestational age and embedded in Matrigel. They were then dyed with three different fluorescent dyes. The dyes were either injected directly into a point within the embryo or injected into the Matrigel just outside of the embryo. The dyes were then allowed to diffuse for a short time through the embryo cells, resulting in either a small locus of dyed cells (if the dye was injected into the embryo; FIG. 6B), or an extended region of dyed cells (if the dye was injected into the Matrigel; FIG. 6C). The embryo was then photographed, (in visible light and all three fluorescent channels) and dissociated into a single cell suspension. Finally, the embryo cells were FACS sorted (with index-sorting) into 384-well MARS plates, and their single-cell RNA profiles recovered using MARS-seq.

No attempt was made to maintain identical dye injection positions. The fluorescence pattern differed therefore greatly between embryos, depending on the injection position, the exact amount of dye injected and the stochasticity of the diffusion process.

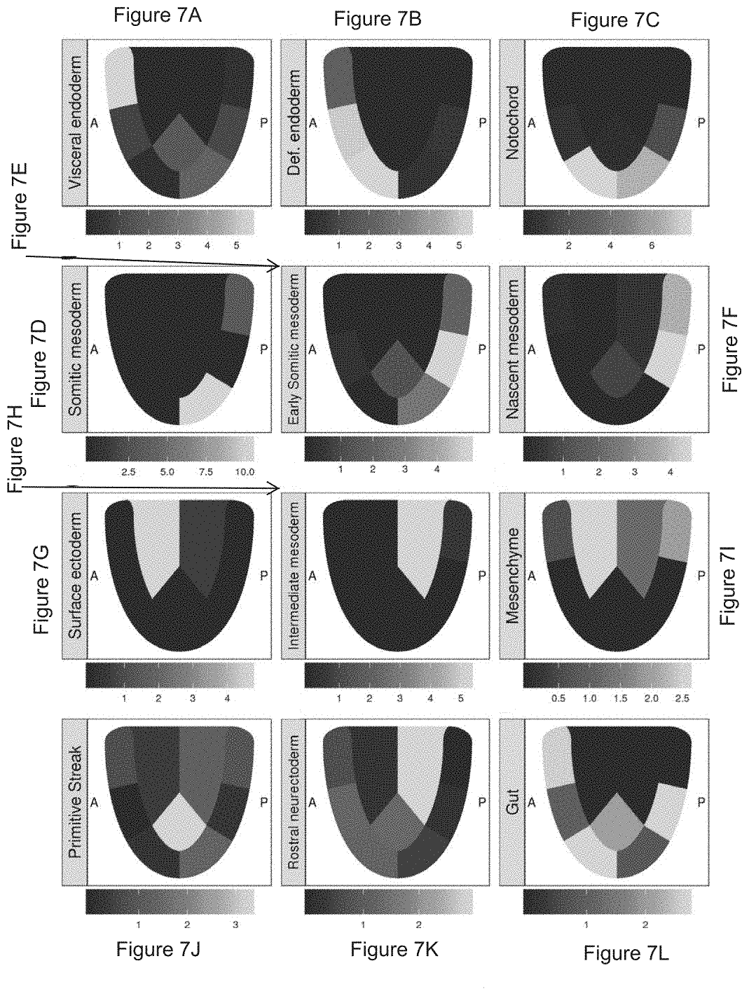

The StainSeq protocol was applied to 18 wild-type E7.5 embryos, collecting a total of 6,457 cells. Based on a preexisting cell atlas, 22 different cell types were detected that were sampled deeply enough to allow the inference of spatial distributions (FIGS. 7A-L). The spatial distribution inferred for most cell types recapitulate known results about the embryo at that gestational age: Remaining visceral endoderm is located at the rostral end (FIG. 7A) giving way to newly created definitive endoderm further along the trunk (FIG. 7B). The notochord is formed at the embryo's midline halfway between the rostral and caudal ends (FIG. 7C). Somitic, early somitic and nascent mesoderm are at the embryo's midline, arrayed towards the caudal end (FIGS. 7D-F). Surface ectoderm is located at the embryo's rostral end (FIG. 7G). Intermediate mesoderm is at the lateral part of the caudal end (FIG. 7H).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Application

1 Baccin, C. et al. Combined single-cell and spatial transcriptomics reveal the molecular, cellular and spatial bone marrow niche organization. Nat Cell Biol 22, 38-48 (2020).
2 Halpern, K. B. et al. Paired-cell sequencing enables spatial gene expression mapping of liver endothelial cells. Nat Biotechnol 36, 962-970 (2018).
3 Kruse, F., Junker, J. P., van Oudenaarden, A. & Bakkers, J. Tomo-seq: A method to obtain genome-wide expression data with spatial resolution. Methods Cell Biol 135, 299-307 (2016).
4 Medaglia, C. et al. Spatial reconstruction of immune niches by combining photoactivatable reporters and scRNA-seq. Science 358, 1622-1626 (2017).
Pijuan-Sala, B. et al. A single-cell molecular map of mouse gastrulation and early organogenesis. Nature 566, 490-495 (2019).
6 Rodriques, S. G. et al. Slide-seq: A scalable technology for measuring genome-wide expression at high spatial resolution. Science 363, 1463-1467 (2019).
7 Wu, C. C. et al. Spatially Resolved Genome-wide Transcriptional Profiling Identifies BMP Signaling as Essential Regulator of Zebrafish Cardiomyocyte Regeneration. Dev Cell 36, 36-49 (2016).
8 Asp, M. et al. A Spatiotemporal Organ-Wide Gene Expression and Cell Atlas of the Developing Human Heart. Cell 179, 1647-1660 e1619 (2019).
9 Codeluppi, S. et al. Spatial organization of the somatosensory cortex revealed by osmFISH. Nat Methods 15, 932-935 (2018).
Halpern, K. B. et al. Single-cell spatial reconstruction reveals global division of labour in the mammalian liver. Nature 542, 352-356 (2017).
11 Satija, R., Farrell, J. A., Gennert, D., Schier, A. F. & Regev, A. Spatial reconstruction of single-cell gene expression data. Nat Biotechnol 33, 495-502 (2015).
12 Farrell, J. A. et al. Single-cell reconstruction of developmental trajectories during zebrafish embryogenesis. Science 360 (2018).
13 Nitzan, M., Karaiskos, N., Friedman, N. & Rajewsky, N. Gene expression cartography. Nature 576, 132-137 (2019).
14 Askary, A. et al. In situ readout of DNA barcodes and single base edits facilitated by in vitro transcription. Nat Biotechnol 38, 66-75 (2020).
Peng, G. et al. Molecular architecture of lineage allocation and tissue organization in early mouse embryo. Nature 572, 528-532 (2019).
16 Serizawa, T. et al. Developmental analyses of mouse embryos and adults using a non-overlapping tracing system for all three germ layers. Development 146 (2019).

What is claimed is:

1. A method of identifying a molecular composition and a spatial position of a single cell comprised in a 3 dimensional (3D) structure comprising a plurality of cells, the method comprising:
(a) injecting into the 3D structure at least one dye to form an identifiable color pattern of said at least one dye over said plurality of cells;
(b) identifying said color pattern in said 3D structure so as to index said plurality of cells according to spatial positions of said cells in said 3D structure;
(c) isolating cells from said 3D structure comprising said at least one dye to obtain single cells;
(d) determining a molecular composition and staining of said single cells;
(e) aligning said staining of said single cells to said index so as to identify the spatial position of the single cells.

2. The method of claim 1, wherein said color pattern comprises a diffusion gradient.

3. The method of claim 1, wherein said color pattern comprises a plurality of colors, each characterized by a different hue or central wavelength.

4. The method of claim 1, wherein the plurality of cells comprise a tissue, an organoid, an organ or an organism.

5. The method of claim 1, wherein the 3D structure comprises a gel embedding said plurality of cells.

6. The method of claim 1, wherein said dye is at least one of:
(i) non-toxic;
(ii) membrane permeable;
(iii) capable of forming staining diffusion gradient; and
(iv) not leaking from cells following said isolating.

7. The method of claim 1, wherein said staining diffusion gradient is obtained by a plurality of dyes.

8. The method of claim 1, wherein said staining diffusion gradient is obtained by varying concentrations of said at least one dye.

9. The method of claim 7, wherein said staining diffusion gradient is an opposing gradient or coalescing gradient.

10. The method of claim 1, wherein said staining diffusion gradient is a radial gradient.

11. A method of identifying a position of a single cell in an image of cells, the method comprising:
receiving a staining characteristic of the single cell;
identifying a color pattern in the image, so as to index said cells according to spatial positions of said cells in image; and
aligning said staining of the single cell to said index so as to identify the spatial position of the single cell.

12. The method according to claim 11, wherein said color pattern comprises a diffusion gradient.

13. The method according to claim 11, wherein said color pattern comprises a plurality of colors, each characterized by a different hue or central wavelength.

14. The method according to claim 11, wherein said identifying said color pattern, comprises thresholding picture-elements in the image, to binary classify each picture-element as stained or non-stained.

15. The method according to claim 11, being executed for a plurality of cell types, wherein said aligning comprises estimating a likelihood for the plurality of cell types to have a respective plurality of staining characteristics.

16. The method according to claim 15, further comprising applying an optimization procedure to said estimated likelihood.

17. The method according to claim 16, wherein said optimization procedure is a non-linear optimization procedure.

18. The method according to claim 16, wherein said optimization procedure comprises Monte-Carlo simulation.

19. A computer software product, comprising a computer-readable medium in which program instructions are stored, which instructions, when read by a data processor, cause the data processor to receive an image of cells, and a staining characteristic of a single cell, and to execute the method according to claim 11.

20. A system for identifying a position of a single cell in an image of cells, the system comprising:
an input circuit receiving an image of cells, and a staining characteristic of the single cell; and
a data processor configured for executing the method according to claim 11.
